(19) United States
(12) Patent Application Publication
(10) Pub. No.: US 2021/0146353 A1
(43) Pub. Date: May 20, 2021

(54) A SYSTEM AND A METHOD FOR IRRADIATING BIOLOGICAL MATERIAL

(71) Applicant: BIOGENIUM MICROSYSTEMS OY, Tampere (FI)

(72) Inventors: Pasi KALLIO, TAMPERE (FI); Krista RANTANEN, SALO (FI); Joose KREUTZER, TAMPERE (FI); Olli TANHUANPÄÄ, TAMPERE (FI); Panu JAAKKOLA, Turku (FI)

(21) Appl. No.: 16/493,911

(22) PCT Filed: Mar. 12, 2018
(86) PCT No.: PCT/FI2018/050176

§ 371 Date: Sep. 13, 2019

(30) Foreign Application Priority Data
Mar. 14, 2017 (FI) 20175229

(51) Int. Cl. B01L 3/00; C12M 3/00; C12M 1/32; C12M 1/34; C12M 1/00; C12M 1/42

(52) U.S. Cl. CPC B01L 3/50855; C12M 23/42; C12M 23/12; C12M 41/34; B01L 2300/0829; C12M 35/02; B01L 2300/048; B01L 2300/0681; C12M 23/22

(57) ABSTRACT

A system for irradiating biological material includes a platform for holding the biological material and a radiation source for directing X-ray radiation to the biological material. The platform includes platform elements each including a chamber for containing the biological material. The platform includes a frame structure for mechanically supporting the platform elements and for mechanically supporting a gas-supply system for supplying gas to the platform elements. The gas-supply system can be for example a gas cartridge. The frame structure includes gas channels for receiving the gas from the gas-supply system and for conducting the gas to the platform elements so as to provide a desired gas composition in the chambers during irradiation of the biological material.

### FIELD OF THE DISCLOSURE

The disclosure relates to a system and to a method for irradiating biological material in a controlled gas environment. The biological material can be, for example but not necessarily, a cell culture or a sample of tissue extracted from a living organism.

### BACKGROUND

In conjunction with many research projects there can be a need to irradiate a cell culture or other biological material in controlled gas environments. For example, there can be a need to irradiate a cell culture with electromagnetic radiation such as e.g. X-rays or ultraviolet rays and/or with particle radiation such as e.g. α- or β-radiation so that the cells are under hypoxic conditions during the irradiation process. Hypoxia research is an example of such research areas that suffer of environmental changes directed to cell cultures. Therefore, studying the effect of radiation on living cells which are under hypoxic conditions requires continuous control and management of the gas environment of the cells being irradiated.

Commercial hypoxia chambers are available but they are very expensive to purchase and maintain, and thus they are not available for all research groups. Moreover, commercial hypoxia chambers are large hoods which do not provide fast dynamic changes in the gas environment of a cell culture. It might take several hours to stabilize the gas environment after a change. Furthermore, it may be challenging to arrange a radiation source to operate inside a hypoxia chamber of the kind mentioned above so that a desired dose of radiation is directed to a cell culture. Thus, there can be a need to move the cell culture out from the hypoxia chamber in order to irradiate the cells. As a corollary, low oxygen concentration disappears rapidly from the gas environment of the cell culture. This might create severe consequences to the cells. Therefore, in this exemplifying research area it is important to keep the cell culture in the same conditions throughout the culture process and also during the irradiation process.

### SUMMARY

The following presents a simplified summary in order to provide a basic understanding of some aspects of various invention embodiments. The summary is not an extensive overview of the invention. It is neither intended to identify key or critical elements of the invention nor to delineate the scope of the invention. The following summary merely presents some concepts of the invention in a simplified form as a prelude to a more detailed description of exemplifying and non-limiting embodiments of the invention.

In accordance with the invention, there is provided a new system for irradiating biological material such as for example a cell culture or a sample of tissue extracted from a living organism. The system comprises:
a platform for holding the biological material, and
a radiation source for directing at least one of the following radiations to the biological material: X-ray radiation, ultraviolet radiation, particle radiation.

The platform comprises one or more platform elements each comprising a chamber for containing the biological material. The platform further comprises a frame structure for mechanically supporting the platform elements and for mechanically supporting a gas-supply system capable of supplying gas to the platform elements without receiving the gas from an external source. The frame structure comprises gas channels for receiving the gas from the gas-supply system and for conducting the gas to the platform elements so as to determine gas composition in each of the chambers during irradiation of the biological material. Each of the platform elements comprises:
a first reservoir for containing liquid-form culturing medium,
a first liquid duct for conducting the liquid-form culturing medium from the first reservoir to the chamber of the platform element under consideration, and
a second liquid duct for conducting the liquid-form culturing medium out from the chamber of the platform element under consideration.

The above-mentioned radiation source can be, for example but not necessarily, a cesium Cs-137 radiation source, an Iridium Ir-192 radiation source, a iodine 1-125 radiation source, a cobalt Co-60 radiation source, or a radium Ra-226 radiation source

The above-described platform and the gas-supply system are capable of constituting a portable device which maintains a desired gas environment of the biological material. Thus, moving the portable device to a place where the irradiation process can be carried out does not cause a change in the gas environment of the biological material. Therefore, the above-described system makes it possible to examine for example how radiation effects on a cell culture under hypoxia conditions.

In accordance with the invention, there is provided also a new method for irradiating biological material such as for example a cell culture or a sample of tissue extracted from a living organism. The method comprises:
holding the biological material in one or more chambers of one or more platform elements of a system according to the invention,
maintaining, in the one or more chambers containing the biological material, a desired gas composition different from that of the ambient air, and
activating the radiation source of the system to direct at least one of the following radiations to the biological material: X-ray radiation, ultraviolet radiation, particle radiation.

A number of exemplifying and non-limiting embodiments of the invention are described in accompanied dependent claims.

Various exemplifying and non-limiting embodiments of the invention both as to constructions and to methods of operation, together with additional objects and advantages thereof, will be best understood from the following description of specific exemplifying embodiments when read in connection with the accompanying drawings.

The verbs “to comprise” and “to include” are used in this document as open limitations that neither exclude nor require the existence of also un-recited features. The features recited in dependent claims are mutually freely combinable unless otherwise explicitly stated. Furthermore, it is to be understood that the use of “a” or “an”, i.e. a singular form, throughout this document does not exclude a plurality.

### BRIEF DESCRIPTION OF FIGURES

Exemplifying and non-limiting embodiments of the invention and their advantages are explained in greater detail below with reference to the accompanying drawings, in which:

FIG. 1a illustrates the mechanical structure of a system according to an exemplifying and non-limiting embodiment of the invention for irradiating biological material,

FIG. 1b illustrates the operation of a system according to an exemplifying and non-limiting embodiment of the invention for irradiating biological material, FIGS. 2a-2g illustrate the mechanical structure of platform elements of system illustrated in FIG. 1a, and

FIG. 3 shows a flowchart of a method according to an exemplifying and non-limiting embodiment of the invention for irradiating biological material.

### DESCRIPTION OF EXEMPLIFYING AND NON-LIMITING EMBODIMENTS

The specific examples provided in the description given below should not be construed as limiting the scope and/or the applicability of the appended claims.

Lists and groups of examples provided in the description given below are not exhaustive unless otherwise explicitly stated.

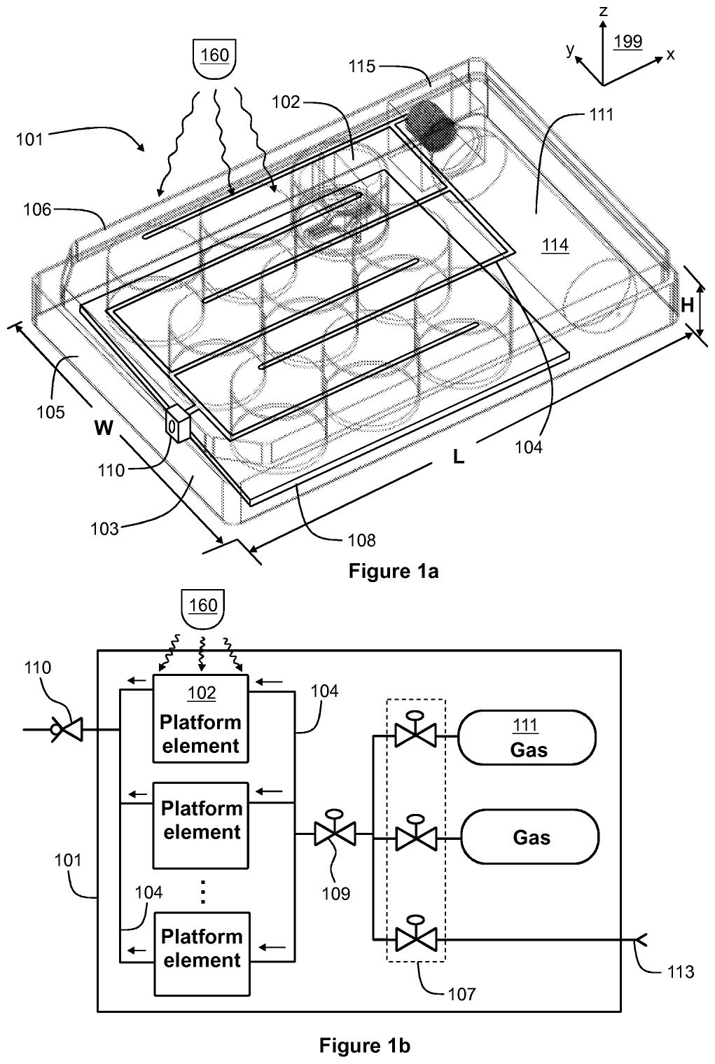

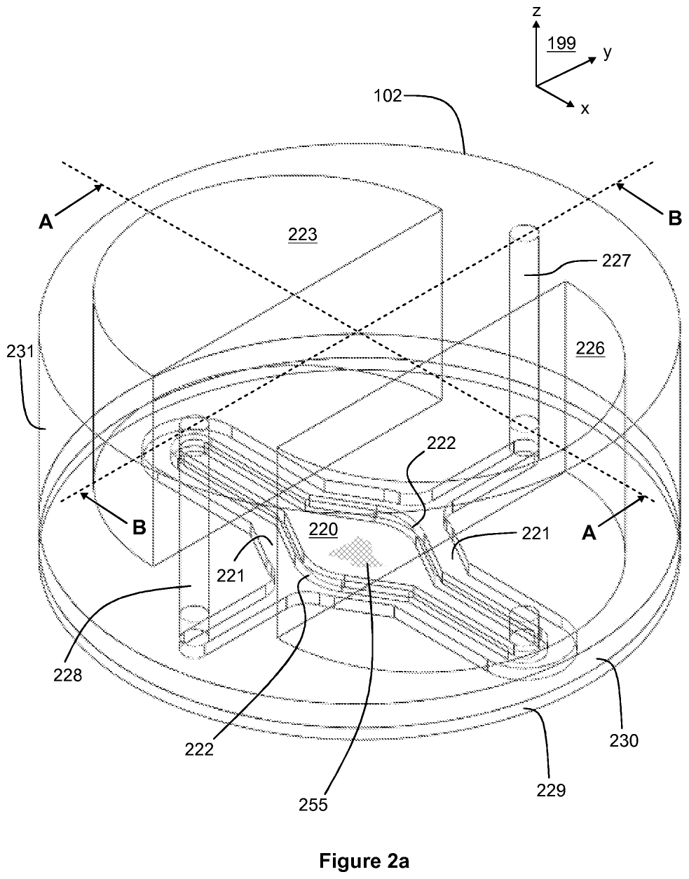

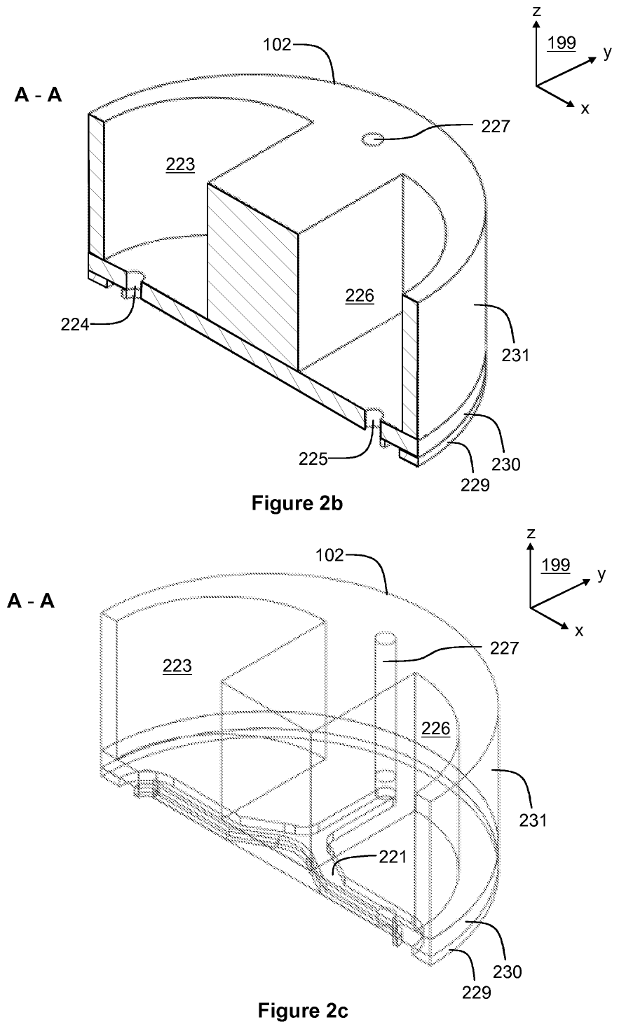

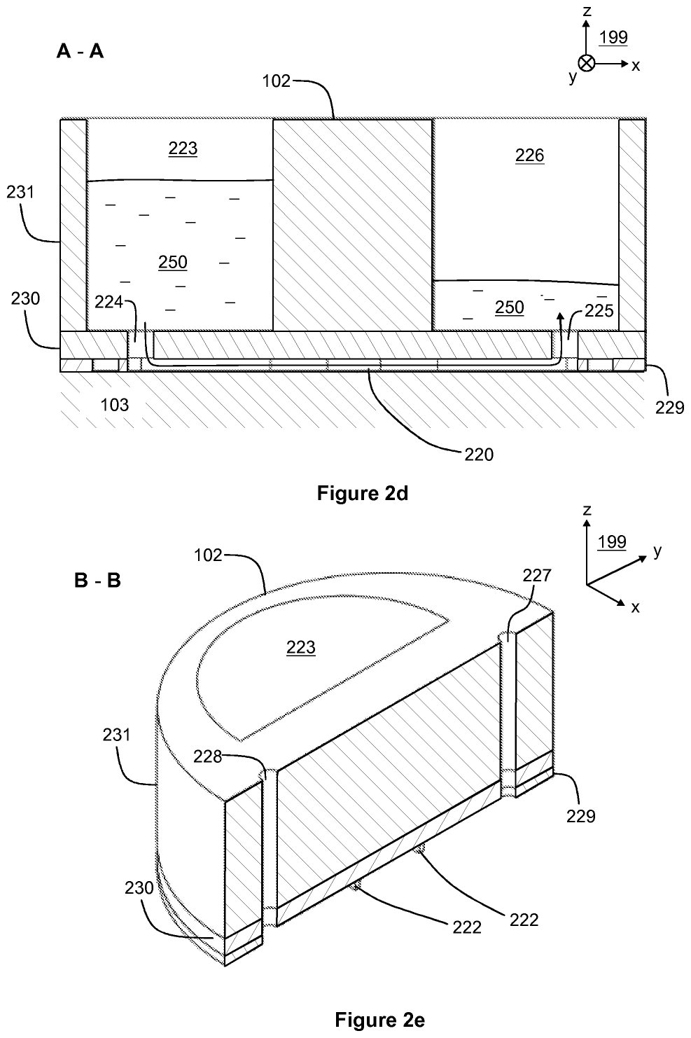

FIG. 1a shows the mechanical structure of a system according to an exemplifying and non-limiting embodiment of the invention for irradiating biological material. The system comprises a platform 101 and a gas-supply system 111 that is capable of supplying gas having a desired composition without receiving the gas from an external source. The system comprises a radiation source 160 for directing X-ray radiation to the biological material. In addition to or instead of X-rays, the radiation source can emit e.g. visible light, ultraviolet “UV” radiation, or some other electromagnetic radiation, and/or particle radiation such as e.g. α- or β-radiation. The platform 101 comprises platform elements each of which comprises a chamber for containing the biological material. In FIG. 1a, one of the platform elements is denoted with a figure reference 102. The other platform elements are not shown in FIG. 1a. The platform 101 further comprises a frame structure 103 for mechanically supporting the platform elements and the gas-supply system 111. The frame structure 103 comprises gas channels 104 for receiving the gas from the gas-supply system 111 and for conducting the gas to the platform elements. In this exemplifying case, the platform elements, such as the platform element 102, are separate components with respect to the frame structure 103. It is however also possible that each platform element is an integral part of the frame structure. The platform elements can be made of for example polydimethylsiloxane “PDMS”.

In the exemplifying system illustrated in FIG. 1a, the platform 101 is suitable for acting as a cell culture platform and each of the platform elements is suitable for acting as a culture element where the chamber for containing biological material is suitable for acting as a culture chamber for containing cultured cells and liquid form culturing medium. Each of the platform elements may comprise a gas room for containing the above-mentioned gas and gas permeable material between the gas room and the chamber for containing biological material. The gas channels 104 are advantageously arranged to conduct the gas to flow through the platform elements so that the gas in the platform elements can be changed rapidly if needed.

The frame structure 103 is advantageously at least partly made of one or more transparent materials. Correspondingly, each platform element is advantageously at least partly made of one or more transparent materials. In this exemplifying case, the biological material can be imaged and/or examined using optical microscopy techniques. The one or more transparent materials may comprise for example polystyrene, polystyrene with copolymers, polyvinyl chloride, polyvinyl chloride with copolymers, polyethylene, polystyrene-acrylonitrile, polypropylene, polyvinylidine chloride, polycarbonate, cyclic olefin copolymer, and/or silicone elastomer.

In the exemplifying platform 101 illustrated in FIG. 1a, the frame structure 103 comprises a well plate 105 and a lid 106. The well plate 105 comprises wells each being capable of containing one of the platform elements, and the lid 106 comprises the above-mentioned gas channels 104. The lid 106 closes the wells when the lid is placed on top of the well plate as illustrated in FIG. 1a. The physical dimensions of the well plate 105 are advantageously similar to the physical dimensions of a standard laboratory well plate so that the platform 101 can be easily placed on e.g. a sample table of a microscope and/or on another instrument. The length L of the platform 101 can be e.g. on the range from 86 mm to 128 mm, the width W of the platform 101 can be e.g. on the range from 60 mm to 86 mm, and the height H of the platform 101 can be e.g. on the range from 18 mm to 45 mm.

In the exemplifying system illustrated in FIG. 1a, the gas-supply system 111 comprises a replaceable container 114 that contains pressurized gas. The replaceable container 114 can be for example a gas cartridge. The replaceable container 114 may contain for example 12 g gas the volume of which is about 7 liters in the normal temperature and pressure “NTP”. With a suitable design of the platform 101, the above-mentioned volume can be enough to maintain sufficient gas flows through individual platform elements for several days. The platform elements are advantageously designed so that a small gas flow rate, e.g. about 100 μl/min, is enough to maintain the desired gas environment inside the platform elements. Using the replaceable container 114 it is not necessary to use a gas pipe connected to an external gas supply system. Therefore, the platform 101 and the gas-supply system 111 constitute a portable device where the gas environment of biological material can be maintained because the gas-supply system 111 is capable of operating autonomously without connections to an external gas supply system. Moving the portable device to a place where an irradiation process can be carried out does not cause a change in the gas environment of the biological material. Therefore, it is possible to examine, for example, how X-ray radiation effects on a cell culture under hypoxia conditions. Furthermore, for example microscopic imaging that may be needed for monitoring the biological material does not cause a change in the gas environment of the biological material. In the exemplifying case illustrated in FIG. 1a, the frame structure 103 comprises a cavity for the replaceable container 114. It is, however, also possible that the frame structure 103 comprises means for fastening a container for pressurized gas to an outer surface of the frame structure.

It is worth noting that the above-mentioned replaceable container 114 is not the only possible choice for the gas-supply system 111. It is also possible that the gas-supply system 111 comprises a refillable container for pressurized gas. The refillable container comprises a refilling valve for enabling the refilling from an external source such as a gas bottle and/or a gas mixer. Furthermore, it is also possible that the gas-supply system comprises a room for containing liquids which interact so that desired gas is generated.

The gas flow in a system according to an exemplifying and non-limiting embodiment of the invention is illustrated in FIG. 1b. The platform 101 of the system comprises the gas channels 104 for receiving the gas from the gas-supply system 111 and for conducting the gas to flow through the platform elements one of which is denoted with the figure reference 102. The platform 101 may comprise a controllable valve 109 for controlling the gas flow through the platform elements. In the exemplifying system illustrated in FIG. 1a, the controllable valve can be located in an element 115. The platform 101 may further comprise a backflow barrier for preventing the ambient air from flowing into the platform elements in a direction opposite to the flowing direction of the gas supplied by the gas-supply system. In FIGS. 1a and 1b, the backflow barrier is denoted with a figure reference 110.

In a system according to an exemplifying and non-limiting embodiment of the invention, the frame structure 103 is configured to mechanically support two or more autonomous gas-supply systems. In this exemplifying case, the gas channels may comprise a selection valve system for selecting which one of the gas-supply systems is enabled to supply gas to the platform elements. In FIG. 1b, the selection valve system is denoted with a figure reference 107. With for example two replaceable containers, e.g. gas cartridges, carried by the frame structure it is possible to maintain a desired gas environment of the biological material during a change of a replaceable container. Furthermore, one of the gas-supply systems may contain different gas than another of the gas-supply systems.

In this exemplifying case, it is possible to alter the gas environment of the biological material in a controlled way. For example, in conjunction with hypoxia research, it is possible to perform reoxygenation experiments so that two different gas concentrations are supplied in relays.

In a system according to an exemplifying and non-limiting embodiment of the invention, the gas channels comprise a pipe interface 113 for connecting to an external gas supply system. In this exemplifying case, an external gas bottle and/or a gas mixer can be connected to the platform 101 with a gas pipe in order to avoid consuming the gas from a replaceable and/or refillable container, e.g. a gas cartridge. In the exemplifying system illustrated in FIG. 1 a, a pipe interface for connecting to an external gas supply system can be located in the element 115.

A system according to an exemplifying and non-limiting embodiment of the invention comprises an electrically operated heating element for controlling temperature of biological material contained by the above-mentioned platform elements. In the exemplifying system illustrated in FIG. 1a, the heating element is denoted with a figure reference 108. The heating element 108 may comprise for example resistor wires which are advantageously so thin that they do not disturb for example microscope imaging and other optical operations. The heating element 108 can be for example battery operated, and the frame structure 103 can be arranged to mechanically support the battery.

FIG. 2a illustrates the platform element 102 shown in FIG. 1a. FIGS. 2b, 2c and 2d show a section obtained by cutting the platform element 102 along a line A-A shown in FIG. 2a. The section plane related to FIGS. 2b-2d is parallel with the xz-plane of a coordinate system 199. In FIGS. 2b and 2d, the section surfaces are denoted with diagonal hatchings. FIGS. 2e, 2f, and 2g show a section obtained by cutting the platform element 102 along a line B-B shown in FIG. 2a. The section plane related to FIGS. 2e-2g is parallel with the yz-plane of the coordinate system 199. The viewing directions related to FIGS. 1a and 2a-2g are illustrated by the coordinate system 199.

The platform element 102 comprises a chamber that is suitable for containing e.g. a cell culture and liquid-form culturing medium. The chamber is denoted with a figure reference 220 in FIGS. 2a, 2d, 2f, and 2g. In FIG. 2a, the cell culture is depicted with a cross-hatched area 255. The platform element 102 comprises a gas room for containing the gas which is used for providing the desired gas environment for the cell culture. The gas room is denoted with a figure reference 221 in FIGS. 2a, 2c, 2f, and 2g. The platform element 102 comprises gas permeable material between the gas room 221 and the chamber 220. The gas permeable material is denoted with a figure reference 222 in FIGS. 2a, 2e, and 2f. The gas permeable material may comprise for example polydimethylsiloxane “PDMS”.

The gas room 221 and the chamber 220 are advantageously separated from each other with water impermeable structures which prevent water from leaking from the chamber to the gas room. An advantage achieved with the water impermeable structures is that the gas can be kept dry, and dry gas makes it possible to use lower gas flow rates and thereby to save gas. Furthermore, the water tight chamber is capable of effectively protecting a cell culture against contaminations.

The exemplifying platform element 102 further comprises a first reservoir for containing the liquid-form culturing medium, a first liquid duct for conducting the liquid-form culturing medium from the first reservoir to the chamber 220, a second liquid duct for conducting the liquid-form culturing medium out from the chamber 220, and a second reservoir 226 connected to the second liquid duct. The above-mentioned first reservoir is denoted with a figure reference 223 in FIGS. 2a-2f, the above-mentioned first liquid duct is denoted with a figure reference 224 in FIGS. 2b and 2d, the above-mentioned second liquid duct is denoted with a figure reference 225 in FIGS. 2b and 2d, and the above-mentioned second reservoir is denoted with a figure reference 226 in FIGS. 2a-2d. In FIG. 2d, the liquid-form culturing medium is denoted with a figure reference 250 and the flow of the liquid-form culturing medium from the first reservoir 223 through the culture chamber 220 to the second reservoir 226 is depicted with a curved line arrow. In the exemplifying situation shown in FIG. 2d, the flow is based on a hydrostatic pressure difference between the first and second reservoirs 223 and 226. As can be seen from FIG. 2d, the frame structure 103 constitutes the bottom for the platform element 102. It is, however, also possible that the platform element 102 has its own bottom element.

The exemplifying platform element 102 further comprises a first gas duct for conducting the gas from the gas channels 104 shown in FIGS. 1a and 1b to the gas room 221 and a second gas duct for conducting the gas out from the gas room 221. The first gas duct is denoted with a figure reference 227 in FIGS. 2a, 2b, 2c, 2e, 2f and 2g, and the second gas duct is denoted with a figure reference 228 in FIGS. 2a, 2e, 2f, and 2g. In FIGS. 2f and 2g, the gas flow is depicted with curved line arrows.

As illustrated in FIG. 2a, the gas room 221 of the platform element 102 surrounds the chamber 220, and a wall between the gas room and the culture chamber comprises the gas permeable material 222. As shown in FIG. 2a, the gas room has an elongated shape so that the gas room is actually a gas channel and thus the volume of the gas room 221 can be relatively small. The small volume of the gas room facilitates making fast changes, if needed, in the gas environment of the cell culture or other biological material. For example, the volume of the gas room 221 can be from about 0.5 to about 3 times the volume of the chamber 220 for containing the cell culture or other biological material.

The mechanical structure of the exemplifying platform element 102 illustrated in FIGS. 2a-2g comprises portions stacked on top of each other. In FIGS. 2a-2g, the above-mentioned portions are denoted with figure references 229, 230, and 231. As shown in FIGS. 2a-2g, the portions 229-231 are stacked on each other in the z-direction of the coordinate system 199. As can be seen from FIGS. 2a-2g, each of the portions 229-231 has a constant cross-sectional profile when a section plane is perpendicular to the z-axis of the coordinate system 199.

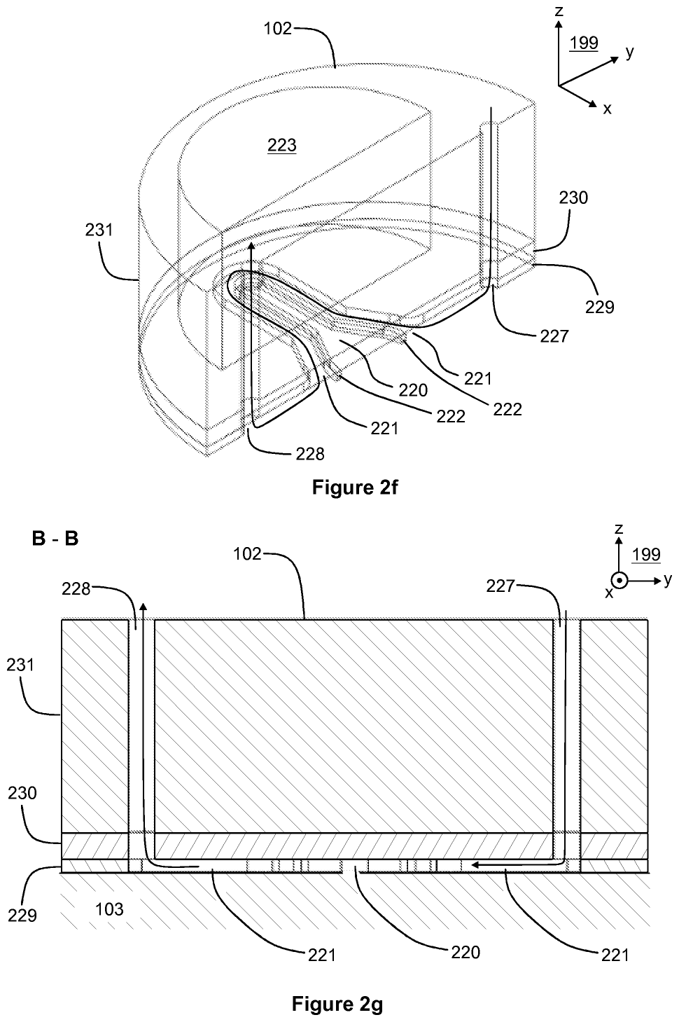

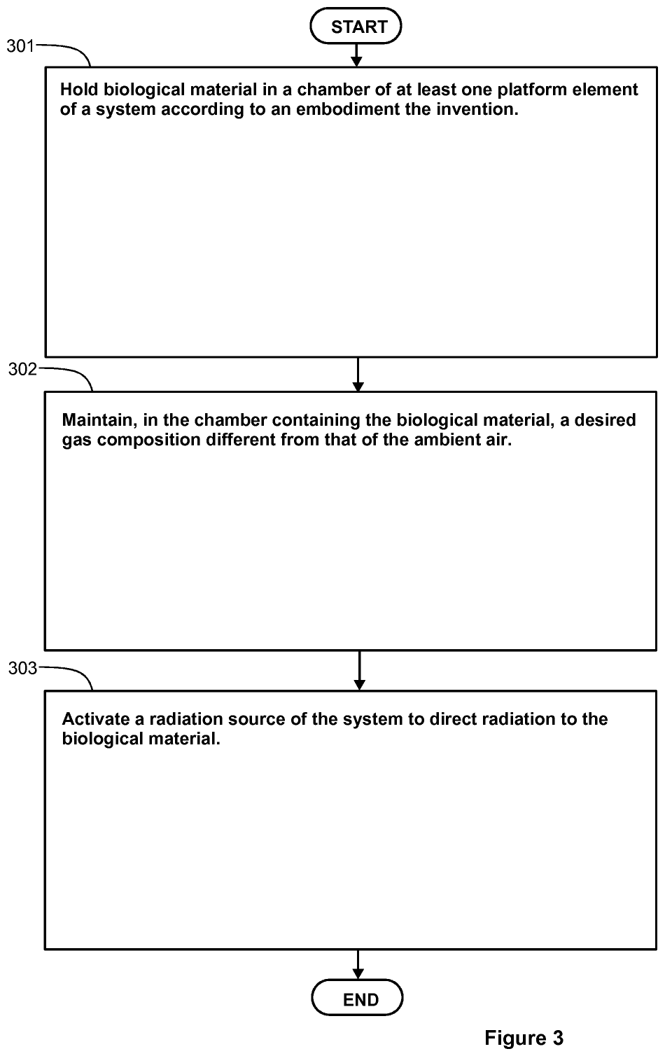

FIG. 3 shows a flowchart of a method according to an exemplifying and non-limiting embodiment of the invention for irradiating biological material such as for example a cell culture or a sample of tissue extracted from a living organism. The method comprises:
action 301: holding the biological material in one or more chambers of one or more platform elements of a system according to an embodiment of the invention,
action 302: maintaining, in the one or more chambers containing the biological material, a gas composition different from that of the ambient air, and
action 303: activating the radiation source of the system to direct X-ray radiation to the biological material.

In a method according to an exemplifying and non-limiting embodiment of the invention, the gas contains less oxygen than the ambient air so as to create hypoxia conditions for the biological material.

In a method according to an exemplifying and non-limiting embodiment of the invention, the system comprises an electrically operated heating element and the temperature of the biological material is controlled with the aid of the electrically operated heating element.

In a method according to an exemplifying and non-limiting embodiment of the invention, the platform elements and a frame structure mechanically supporting the platform elements are at least partly made of one or more transparent materials, and the biological material is optically imaged and/or otherwise inspected with the aid of a microscope.

In a method according to an exemplifying and non-limiting embodiment of the invention, the biological material comprises one or more cell cultures cultured in the above-mentioned system.

The non-limiting, specific examples provided in the description given above should not be construed as limiting the scope and/or the applicability of the appended claims. Lists and groups of examples provided in the description given above are not exhaustive unless otherwise explicitly stated.

1-30. (canceled)

31. A system for irradiating biological material, the system comprising:
a platform for holding the biological material, and
a radiation source for directing at least one of the following radiations to the biological material: X-ray radiation, ultraviolet radiation, particle radiation,
wherein the platform comprises one or more platform elements each comprising a chamber for containing the biological material, wherein the platform further comprises a frame structure for mechanically supporting the platform elements and for mechanically supporting a gas-supply system capable of supplying gas to the platform elements without receiving the gas from an external source, and the frame structure comprises gas channels for receiving the gas from the gas-supply system and for conducting the gas to the platform elements so as to determine gas composition in each of the chambers during irradiation of the biological material, and wherein each of the platform elements comprises:
a first reservoir for containing liquid-form culturing medium,
a first liquid duct for conducting the liquid-form culturing medium from the first reservoir to the chamber of the platform element under consideration, and
a second liquid duct for conducting the liquid-form culturing medium out from the chamber of the platform element under consideration.

32. A system according to claim 31, wherein each of the platform elements comprises a gas room for containing the gas and gas permeable material between the gas room and the chamber.

33. A system according to claim 32, wherein the gas room of each of the platform elements surrounds the chamber of the platform element under consideration, and a wall between the gas room and the chamber comprises the gas permeable material.

34. A system according to claim 32, wherein each of the platform elements comprises:
a first gas duct for conducting the gas from the gas channels to the gas room of the platform element under consideration, and
a second gas duct for conducting the gas out from the gas room of the platform element under consideration.

35. A system according to claim 32, wherein a volume of the gas room of each of the platform elements is from 0.5 to 3 times, preferably from 0.5 to 1 times, a volume of the chamber of the platform element under consideration.

36. A system according to claim 32, wherein the gas permeable material comprises polydimethylsiloxane.

37. A system according to claim 32, wherein the gas room and the chamber are separated from each other with water impermeable structures for preventing water from leaking from the chamber to the gas room.

38. A system according to claim 31, wherein the gas channels are arranged to conduct the gas to flow through the platform elements.

39. A system according to claim 38, wherein the platform further comprises a backflow barrier for preventing ambient air from flowing into the platform elements in a direction opposite to a flowing direction of the gas.

40. A system according to claim 31, wherein the platform is a cell culture platform and each of the platform elements is a culture element in which the chamber for containing the biological material is a culture chamber for containing a cell culture and the liquid form culturing medium.

41. A system according to claim 31, wherein the frame structure comprises a well plate comprising wells each being capable of containing one of the platform elements, and a lid comprising the gas channels and for closing the wells when being placed on top of the well plate.

42. A system according to claim 41, wherein the well plate comprises a cavity for containing the gas-supply system.

43. A system according to claim 31, wherein the frame structure is configured to mechanically support another gas-supply system, and the gas channels comprise a selection valve system for selecting which one of the gas-supply systems is enabled to supply the gas to the platform elements.

44. A system according to claim 31, wherein each of the platform elements comprises a second reservoir connected to the second liquid duct of the platform element under consideration.

45. A system according to claim 31, wherein each of the platform elements comprises portions stacked on top of each other such that each of the portions has a constant cross-sectional profile when a section plane is perpendicular to a direction in which the portions are on top of each other.

46. A system according to claim 31, wherein the frame structure constitutes a bottom for each of the platform elements.

47. A system according to claim 31, wherein the frame structure and the platform elements are at least partly made of one or more transparent materials so as to enable optical inspection of the biological material.

48. A system according to claim 47, wherein the one or more transparent materials comprise one or more of the following: polystyrene, polystyrene with copolymers, polyvinyl chloride, polyvinyl chloride with copolymers, polyethylene, polystyrene-acrylonitrile, polypropylene, polyvinylidine chloride, silicone elastomer.

49. A system according to claim 31, wherein the platform further comprises an electrically operated heating element for controlling temperature of the biological material.

50. A system according to claim 31, wherein the platform further comprises a controllable valve for controlling a flow rate of the gas to the platform elements.

51. A system according to claim 31, wherein the system comprises the gas-supply system and the gas-supply system is mechanically supported by the frame structure of the platform.

52. A system according to claim 51, wherein the gas-supply system comprises a replaceable container for pressurized gas.

53. A system according to claim 51, wherein the gas-supply system comprises a refillable container for pressurized gas, the refillable container comprising a refilling valve.

54. A system according to claim 31, wherein the platform comprises a pipe interface for receiving the gas from an external source.

55. A method for irradiating biological material, the method comprising:
holding the biological material in one or more chambers of one or more platform elements of a system for irradiating the biological material,
maintaining, in the one or more chambers containing the biological material, a gas composition different from that of ambient air, and
activating the radiation source of the system to direct at least one of the following radiations to the biological material: X-ray radiation, ultraviolet radiation, particle radiation,
wherein the system comprises a platform that comprises the one or more platform elements each comprising one of the one or more chambers, wherein the platform further comprises a frame structure mechanically supporting the platform elements and mechanically supporting a gas-supply system supplying gas to the platform elements without receiving the gas from an external source, and the frame structure comprises gas channels receiving the gas from the gas-supply system and conducting the gas to the platform elements so as to determine the gas composition in each of the chambers during irradiation of the biological material, and
wherein each of the platform elements comprises:
a first reservoir for containing liquid-form culturing medium,
a first liquid duct for conducting the liquid-form culturing medium from the first reservoir to the chamber of the platform element under consideration, and
a second liquid duct for conducting the liquid-form culturing medium out from the chamber of the platform element under consideration.

56. A method according to claim 55, wherein the gas contains less oxygen than the ambient air so as to create hypoxia conditions for the biological material.

57. A method according to claim 55, wherein the method comprises controlling temperature of the biological material with an electrically operated heating element of the platform.

58. A method according to claim 55, wherein the frame structure and the platform elements are at least partly made of one or more transparent materials, and the method comprises optically inspecting the biological material with the aid of a microscope.

59. A method according to claim 55, wherein the platform is a cell culture platform and each of the platform elements is a culture element in which the chamber for containing the biological material is a culture chamber for containing a cell culture and the liquid form culturing medium, and the biological material comprises one or more cell cultures cultured in the system.
